(19) United States
(12) Patent Application Publication
(10) Pub. No.: US 2015/0272930 A1
(43) Pub. Date: Oct. 1, 2015

(54) Fixed Dosage Formulations of Angiotensin Converting Enzyme (ACE) inhibitor and the Diuretic Chlorthalidone

(71) Applicants: Dorai T. Rajan, Parkersburgh, WV (US); Himanshu Sud, Lenexa, KS (US)

(72) Inventors: Dorai T. Rajan, Parkersburgh, WV (US); Himanshu Sud, Lenexa, KS (US)

(73) Assignee: AILEX Pharmaceuticals PVT. LTD., Lenexa, KS (US)

(21) Appl. No.: 14/671,589

(22) Filed: Mar. 27, 2015

Related U.S. Application Data
(60) Provisional application No. 61/973,776, filed on Apr. 1, 2014.

(51) Int. Cl. A61K 31/4035; A61K 9/20; A61K 9/08; A61K 38/05

(52) U.S. Cl. CPC A61K 31/4035; A61K 38/05; A61K 9/20; A61K 9/08

(57) ABSTRACT

The invention describes fixed dosage formulations of an angiotensin converting enzyme (ACE) inhibitor, preferably Lisinopril and the diuretic, preferably Chlorthalidone in the same pharmaceutically acceptable carrier, for example a tablet, capsule or oral suspension and methods of treating hypertension by administering the fixed dosage formulation of Lisinopril and Chlorthalidone to a patient in need thereof.

This application claims priority from U.S. Provisional Application No. 61/973,776 filed on Apr. 1, 2014 and herein incorporated by reference in its entirety.

### FIELD OF THE INVENTION

This invention is directed to fixed dose pharmaceutical compositions comprising an angiotensin converting enzyme (ACE) inhibitor and a diuretic, preferably Lisinopril and Chlorthalidone, and to methods of treating hypertension using both drugs in combination.

### BACKGROUND OF THE INVENTION

Hypertension is widely acknowledged to be one of the most widespread of all illnesses. There are about 60 million cases of hypertension in the U.S. It is believed that approximately one third of all patients with hypertension in the U.S. are adequately treated, one third are treated but not adequately, and one third are not treated.

Angiotensin converting enzyme (ACE) inhibitors are considered a suitable first-step option in the treatment of hypertension in a diversity of patient types. Angiotensin converting enzyme inhibitor (ACE inhibitors) drugs that are approved for the treatment of hypertension include Benazepril (Lotensin), Captopril (Capoten), Enalapril/Enalaprilat (Vasotec oral and injectable), Fosinopril (Monopril), Lisinopril (Zestril and Prinivil), Moexipril (Univasc), Perindopril (Aceon), Quinapril (Accupril), Ramipril (Altace), and Trandolapril (Mavik). The Joint National Committee on the Prevention, Detection, Evaluation, and Treatment of High Blood Pressure (JNC 7), the World Health Organization/International Society of Hypertension, the European Society of Hypertension/European Society of Cardiology, and the Canadian Hypertension Education Program Evidence-Based Recommendations Task Force currently endorse ACE inhibitors as an option for the first line therapy in patients with essential hypertension, especially in patients with a high coronary disease risk profile, diabetes with renal disease/proteinuria, heart failure, and/or a history of myocardial infarction.

Lisinopril is one of the most prescribed ACE inhibitors for the treatment of hypertension. The compound 1-(N²-[(S)-1-carboxy-3-phenylpropyl]-L-lysyl)-L-proline, having the generic name lisinopril, as well as therapeutically acceptable salts thereof, are described in U.S. Pat. No. 4,374,829 (Merck & Co. Inc.), incorporated herein by reference. In said patent the compound is described in Example 119, and is referred to as N-α-[1 (S)-1-carboxy-3-phenylpropyl]-L-lysyl-L-proline. The divisional application of the '829 patent, which has resulted in U.S. Pat. No. 4,472,380, incorporated herein by reference, claims pharmaceutical compositions that include lisinopril pharmaceutical compositions. Lisinopril is a drug on which extensive clinical experience has been obtained. It is currently sold in the United States under the trademark ZESTRIL® by AstraZeneca or PRINIVIL® by Merck & Co.

A typical lisinopril formulation consists of lisinopril dihydrate, which can be any dose from 1 mg-100 mg, the fillers (diluents)-dibasic calcium phosphate dihydrate and mannitol, maize starch as a binder and disintegrant and magnesium stearate as a lubricant. Dose strength of Lisinopril in FDA approved drug product ranges from 2.5 mg to 40 mg.

The long-acting diuretic chlorthalidone has been known for decades as an effective treatment to lower elevated arterial blood pressure (arterial hypertension or hypertension). The compound received U.S. Pat. No. 3,055,904, which disclosed a dosage range of 50 mg to 200 mg orally one to three times a day or 100 mg every second day. U.S. Pat. No. 5,948,799 discloses a typical range of chlorthalidone of 6.25-200 mg daily and a preferred range of 12.5 to 100 mg daily for use in the treatment of non-ischemic congestive heart failure in combination with amlodipine and/or digoxin; this patent does not address the treatment of hypertension. Chlorthalidone has been chemically described as: benzenesulfonamide, 2-chloro-5-(2,3-dihydro-1-hydroxy-3-oxo-1H-isoindol-lyl) or 2-chloro-5-(1-hydroxy-3-oxo-1,2-dihydroisoindol-1-yl)-benzenesulfonamide.

The U.S. Food and Drug Administration's (FDA) approved starting dose for hypertension for chlorthalidone is 15 mg daily; and the marketed dose strengths are 15, 25 and 50 mg for monotherapy.

It has been recognized since the 1980s that monotherapy does not achieve blood pressure (BP) goals in the majority of patients with hypertension, particularly those with stage 2 hypertension and those with comorbidities, such as diabetes, high coronary disease risk profile, heart failure and/or a history of myocardial infarction. Most patients require 2 or more antihypertensive agents from complementary classes to achieve BP control.

The blood pressure (BP)-lowering ability of an ACE inhibitor is enhanced by the administration of a diuretic, particularly in patients with a salt-sensitive form of hypertension. This type of response has been the basis for the development of fixed-dose combination products consisting of an ACE inhibitor and a thiazide-type diuretic. The underlying principle for combining these two drug classes is that diuretic-induced sodium depletion activates the renin-angiotensin axis [RAA] and moves BP to an angiotensin-Il-dependent mode. FDA approved fixed-dose combination drugs consisting of an ACE inhibitor and hydrochlorothiazide (HCTZ) and their dose strengths (in mg) are below:

### Benazepril/HCTZ 5/6.25; 10/12.5; 20/12.5; 20/25

### Captopril/HCTZ 25/15; 25/25; 50/15; 50/25

### Enalapril/HCTZ 5/12.5; 10/25

### Fosinopril/HCTZ 10/12.5; 20/12.5

### Lisinopril/HCTZ 10/12.5; 20/12.5; 20/25

### Quinapril/HCTZ 10/12.5; 20/12.5; 20/25

### Moexipril/HCTZ 7.5/12.5; 15/12.5; 15/25

Chlorthalidone was extensively studied versus another diuretic, hydrochlorothiazide (HCTZ), in the 1970's, in the Multiple Risk Factor Intervention Trial, a National Institute of Health (NIH)-sponsored study. The investigators concluded that chlorthalidone appeared potentially superior to HCTZ. Subsequently, NIH sponsored four (4) large-scale health outcomes utilizing a diuretic. The diuretic chosen each time was chlorthalidone. The four studies were the Treatment of Mild Hypertension Study (TOMHS), Hypertension Detection and Follow-Up Program (HDFP), Systolic Hypertension in the Elderly Program (SHEP), and the Antihypertensive and Lipid-Lowering Treatment to Prevent Heart Attack Trial (ALLHAT) study.

Despite extensive clinical experiences with chlorthalidone, hydrochlorothiazide (HCTZ) remains the more popular diuretic choice among clinicians in the context of developing single-pill antihypertensive combinations. Chlorthalidone has a longer duration of action compared with HCTZ and is a more effective antihypertensive agent over 24 hours. Because it was used in most of the US-based hypertension outcome trials, including ALLHAT, preferred use of chlorthalidone over HCTZ has been advocated by some authorities.

However, there are no approved fixed-dose combination products containing chlorthalidone and an ACE inhibitor.

### BRIEF DESCRIPTION OF THE INVENTION

The present invention provides for a fixed-dose combination of an Angiotensin Converting Enzyme (ACE) inhibitor and Chlorthalidone (Diuretic). Fixed-dose drug combinations provide potential advantages over monotherapy such as greater BP-lowering effects, use of lower doses of component drugs, the possibility of decreased incidence of clinical and metabolic adverse effects, longer duration of action than component agents, decreased costs and improved patient compliance. Additional benefits to a fixed combination dosage form include lower cost to the patient and greater patient compliance. The fixed combination dosage form can be embodied in a scored or unscored tablet, capsule, pill or dragee. Alternatively, the fixed combination dosage form can be provided in the form of a syrup where the desired ratio of Angiotensin Converting Enzyme inhibitor to Chlorthalidone is provided and the patient takes the prescribed amount of syrup. The syrup would be provided with appropriate flavoring agents, stabilizers, solubility agents and other excipients to form a pharmaceutically acceptable oral syrup.

A preferred Angiotensin Converting Enzyme (ACE) inhibitor is Lisinopril although other ACE inhibitors such as Benazepril, Captopril, Enalapril, Fosinopril, Quinapril and Moexipril can also be used.

### DETAILED DESCRIPTION OF THE INVENTION

The invention may be manufactured as an oral syrup, scored tablet, unscored tablet, or as a capsule. In the most preferred embodiments of the subject invention, the invention is a capsule or a tablet for oral ingestion. Formulations and manufacturing techniques for tablets and capsules are well known in the art, and are described, for example, in Remington's Pharmaceutical Sciences 20th Edition, Mack Publishing Company, Easton, Pa. (2000), which is incorporated herein by reference. Other embodiments of the subject invention may include all of the known modes of administering pharmaceuticals, such as transdermal, buccal, rectal, intravenous, intramuscular, and the like.

In general, lower doses of Lisinopril are preferred to be co-formulated with lower doses of Chlorthalidone, and higher doses of Lisinopril tend to be more useful with higher doses of Chlorthalidone. More preferred daily doses for adults range from 2.5 mg to 100 mg of Chlorthalidone and 2.5 mg to 100 mg of Lisinopril. Most preferred daily dose for adults range from 5 mg to 50 mg of Chlorthalidone and 5 mg to 50 mg of Lisinopril.

Examples of Lisinopril Chlorthalidone formulations per dose are as follows:

about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 10 to 15 milligrams Chlorthalidone;
about 10 to 15 milligrams Lisinopril and about 15 to 20 milligrams Chlorthalidone;
about 10 to 15 milligrams Lisinopril and about 20 to 25 milligrams Chlorthalidone;
about 15 to 20 milligrams Lisinopril and about 10 to 15 milligrams Chlorthalidone;
about 15 to 20 milligrams Lisinopril and about 15 to 20 milligrams Chlorthalidone;
about 15 to 20 milligrams Lisinopril and about 20 to 25 milligrams Chlorthalidone;
about 20 to 25 milligrams Lisinopril and about 10 to 15 milligrams Chlorthalidone;
about 20 to 25 milligrams Lisinopril and about 15 to 20 milligrams Chlorthalidone;
about 20 to 25 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 10 to 15 milligrams Chlorthalidone;
about 25 to 35 milligrams Lisinopril and about 15 to 20 milligrams Chlorthalidone;
about 25 to 35 milligrams Lisinopril and about 20 to 25 milligrams Chlorthalidone;
about 25 to 35 milligrams Lisinopril and about 25 to 30 milligrams Chlorthalidone;
about 25 to 35 milligrams Lisinopril and about 30 to 40 milligrams Chlorthalidone;
about 35 to 50 milligrams Lisinopril and about 10 to 15 milligrams Chlorthalidone;
about 35 to 50 milligrams Lisinopril and about 15 to 20 milligrams Chlorthalidone;
about 35 to 50 milligrams Lisinopril and about 20 to 25 milligrams Chlorthalidone;
about 35 to 50 milligrams Lisinopril and about 25 to 30 milligrams Chlorthalidone;
about 35 to 50 milligrams Lisinopril and about 30 to 40 milligrams Chlorthalidone;
about 35 to 50 milligrams Lisinopril and about 40 to 50 milligrams Chlorthalidone;
about 35 to 50 milligrams Lisinopril and about 65 to 80 milligrams Chlorthalidone;
about 35 to 50 milligrams Lisinopril and about 80 to 100 milligrams Chlorthalidone;
about 50 to 65 milligrams Lisinopril and about 10 to 15 milligrams Chlorthalidone;
about 50 to 65 milligrams Lisinopril and about 15 to 20 milligrams Chlorthalidone;
about 50 to 65 milligrams Lisinopril and about 20 to 25 milligrams Chlorthalidone;
about 50 to 65 milligrams Lisinopril and about 25 to 30 milligrams Chlorthalidone;
about 50 to 65 milligrams Lisinopril and about 30 to 40 milligrams Chlorthalidone;
about 50 to 65 milligrams Lisinopril and about 40 to 50 milligrams Chlorthalidone;
about 50 to 65 milligrams Lisinopril and about 50 to 65 milligrams Chlorthalidone.
about 50 to 65 milligrams Lisinopril and about 65 to 80 milligrams Chlorthalidone;
and about 50 to 65 milligrams Lisinopril and about 80 to 100 milligrams Chlorthalidone.

A preferred example of the dosage of the Lisinopril and Chlorthalidone formulation is:

about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 20 to 25 milligrams Chlorthalidone;
about 15 to 20 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 30 to 40 milligrams Chlorthalidone;
and about 35 to 50 milligrams Lisinopril and about 40 to 50 milligrams Chlorthalidone.

An even more preferred example of the dosage of the Lisinopril and Chlorthalidone formulation is:

about 2.5 milligrams Lisinopril and 6.25 milligrams Chlorthalidone;
about 5 milligrams Lisinopril and about 12.5 milligrams Chlorthalidone;
about 10 milligrams Lisinopril and about 25 milligrams Chlorthalidone;
about 20 milligrams Lisinopril and about 50 milligrams Chlorthalidone;
and about 40 milligrams Lisinopril and about 100 milligrams Chlorthalidone.

In a preferred embodiment of the novel tablet, lisinopril and chlorthalidone are formulated separately with suitable excipients. A suitable inactive segment is formulated, and the tablet is prepared on a tri-layer tablet press, such as a commercially available multi-layer tablet press manufactured by Korsch AG of Germany. Alternatively, lisinopril and chlorthalidone are co-formulated with suitable excipients and compressed in an appropriate layer press with at least one other layer derived from an inactive composition, e.g., granulation. In the standard tablets and capsules well known in the art, a suitable co-formulation is prepared and either tableted or encapsulated, and no separately formulated inactive granulation is utilized. For encapsulation, the individually granulated lisinopril and chlorthalidone granules may either be filled in a single capsule or in a double capsule (capsule inside a capsule; inner capsule containing one drug and the outer capsule the other).

As is seen in the following Example and experimental section, the amount of Chlorthalidone and Lisinopril can be easily varied as can the amounts of the excipients with the only limitation being the need to form an acceptable tablet.

### Example-1

### Preparation of a tri-layer 10 mg Chlorthalidone and 5 mg Lisinopril Tablet

Formulation of Chlorthalidone active blend
The following ingredients are used at the specified weight percentages to formulate a Chlorthalidone active blend composition;

| Ingredient | Quantity, mg | Weight % |
|---|---|---|
| Chlorthalidone | 10.0 | 6.67 |
| Dibasic calcium phosphate, anhydrous | 23.0 | 15.33 |
| Microcrystalline cellulose | 110.6 | 73.73 |
| Sodium starch glycolate | 6.1 | 4.07 |
| Magnesium stearate | 0.3 | 0.2 |
| Total | 150.0 | 100 |

### Steps:

1. Chlorthalidone and an equal mass of microcrystalline cellulose (MCC) are added into a high shear mixer and mixed for 3 minutes.
2. The mixture from Step-1 is placed in a suitably sized “V” blender; the remaining microcrystalline cellulose and sodium starch glycolate are then added and mixed for 15 minutes.
3. Half of the magnesium stearate is added to the above mixture and blended for 3 minutes.
4. The blended mixture from Step-3 is dry granulated on a suitable roller compactor. The roller-compacted material is then milled to a particle size suitable for tablet compression.
5. The milled material from Step-4 is placed in a suitably sized “V” blender. The remaining magnesium stearate is added to the blender and the material is mixed for 3 minutes to obtain the final active blend.

### Formulation of Inactive Blend

The following ingredients are used at the specified weight percentages to formulate an inactive blend composition:

| Ingredient | Quantity, mg | Weight % |
|---|---|---|
| Dibasic calcium phosphate, anhydrous | 26.0 | 17.33 |
| Microcrystalline cellulose | 117.4 | 78.27 |
| Sodium starch glycolate | 6.0 | 4.0 |
| Magnesium stearate | 0.6 | 0.4 |
| Total | 150.0 | 100 |

### Steps:

1. The dibasic calcium phosphate, anhydrous, microcrystalline cellulose, and sodium starch glycolate are added to a suitable “V” blender and mixed for 15 minutes.
2. Half of the magnesium stearate is added to the above mixture and blended for 3 minutes.
3. The blended mixture from Step-2 is dry granulated on a suitable roller compactor. The roller-compacted material is milled to a particle size suitable for tablet compression.
4. The milled material is added to a suitably sized “V” blender. The remaining magnesium stearate is added to the blender and the material is mixed for 3 minutes to obtain the final inactive blend.

### Formulation of Lisinopril Active Blend

The following ingredients are used at the specified weight percentages to formulate a Lisinopril active blend composition;

| Ingredient | Quantity, mg | Weight % |
|---|---|---|
| Lisinopril | 5.0 | 3.33 |
| Dibasic calcium phosphate, anhydrous | 23.0 | 15.33 |
| Microcrystalline cellulose | 115.6 | 77.07 |
| Sodium starch glycolate | 6.1 | 4.07 |
| Magnesium stearate | 0.3 | 0.2 |
| Total | 150.0 | 100 |

### Steps:

1 Lisinopril and an equal mass of microcrystalline cellulose (MCC) are added into a high shear mixer and mixed for 3 minutes.
2 The mixture from Step-1 is placed in a suitably sized “V” blender; the remaining microcrystalline cellulose and sodium starch glycolate are then added and mixed for 15 minutes.
3 Half of the magnesium stearate is added to the above mixture and blended for 3 minutes.
4 The blended mixture from Step-3 is dry granulated on a suitable roller compactor. The roller-compacted material is then milled to a particle size suitable for tablet compression.
5 The milled material from Step-4 is placed in a suitably sized “V” blender. The remaining magnesium stearate is added to the blender and the material is mixed for 3 minutes to obtain the final active blend.
   Tri-Layer Tablet formation

Tablets can be compressed using a multi layer tablet press such as Korsch TRP 900 multi layer tablet press. The inactive layer separates the Chlorthalidone layer and the Lisinopril layer. In this example, all three layers are of equal weight, 150 mg each.

Total tablet weight is 450 mg and the tablet contains 10 mg of Chlorthalidone active and 5 mg of Lisinopril active, respectively.

### Oral Syrup

The co-formulation of Lisinopril and Chlorthalidone as a pharmaceutically acceptable syrup is also part of the invention. The liquid, orally dosable solution of Lisinopril and Chlorthalidone comprises, on a weight to weight basis, about 1 to 2 parts Lisinopril to every 1 to 4 parts Chlorthalidone, in sufficient pharmaceutically acceptable syrup, to provide a pharmaceutically acceptable dose of the Lisinopril-Chlorthalidone combination. The intention is to allow a patient in need thereof to take a liquid dose of Lisinopril and Chlorthalidone appropriate for the treatment, in a convenient 5 to 10 ml or so amount with the absolute amount of Lisinopril and Chlorthalidone being varied so as to give the appropriate dosage of the Lisinopril and Chlorthalidone to a patient in need thereof. The co-formulated syrup should be shelf stable meaning that the components of the formulation should not settle and the syrup will not lose potency or support bacterial contamination.

Other stable liquid suspensions of Lisinopril and Chlorthalidone with the following ratios can be made, where the dosage form is selected from:

about 2 to 5 milligrams Lisinopril and about 2 to 5 milligrams Chlorthalidone in 5 milliliter suspension;
about 2 to 5 milligrams Lisinopril and about 5 to 10 milligrams Chlorthalidone in 5 milliliter suspension;
about 5 to 10 milligrams Lisinopril and about 5 to 10 milligrams Chlorthalidone in 5 milliliter suspension;
about 5 to 10 milligrams Lisinopril and about 10 to 15 milligrams Chlorthalidone in 5 milliliters suspension;
about 10 to 15 milligrams Lisinopril and about 10 to 15 milligrams Chlorthalidone in 5 milliliter suspension;
about 10 to 15 milligrams Lisinopril and about 15 to 20 milligrams Chlorthalidone in 5 milliliter suspension;
about 10 to 15 milligrams Lisinopril and about 20 to 25 milligrams Chlorthalidone in 5 milliliter suspension;
about 15 to 20 milligrams Lisinopril and about 10 to 15 milligrams Chlorthalidone in 5 milliliter suspension;
about 15 to 20 milligrams Lisinopril and about 15 to 20 milligrams Chlorthalidone in 5 milliliter suspension;
about 15 to 20 milligrams Lisinopril and about 20 to 25 milligrams Chlorthalidone in 5 milliliter suspension;
about 20 to 25 milligrams Lisinopril and about 10 to 15 milligrams Chlorthalidone in 5 milliliter suspension;
about 20 to 25 milligrams Lisinopril and about 15 to 20 milligrams Chlorthalidone in 5 milliliter suspension;
about 20 to 25 milligrams Lisinopril and about 20 to 25 milligrams Chlorthalidone in 5 milliliter suspension;
about 20 to 25 milligrams Lisinopril and about 25 to 30 milligrams Chlorthalidone in 5 milliliter suspension;
about 25 to 35 milligrams Lisinopril and about 10 to 15 milligrams Chlorthalidone in 5 milliliter suspension;
about 25 to 35 milligrams Lisinopril and about 15 to 20 milligrams Chlorthalidone in 5 milliliter suspension;
about 25 to 35 milligrams Lisinopril and about 20 to 25 milligrams Chlorthalidone in 5 milliliter suspension;
about 25 to 35 milligrams Lisinopril and about 25 to 30 milligrams Chlorthalidone in 5 milliliter suspension;
about 25 to 35 milligrams Lisinopril and about 30 to 40 milligrams Chlorthalidone in 5 milliliter suspension;
about 35 to 50 milligrams Lisinopril and about 10 to 15 milligrams Chlorthalidone in 5 milliliter suspension;
about 35 to 50 milligrams Lisinopril and about 15 to 20 milligrams Chlorthalidone in 5 milliliter suspension;
about 35 to 50 milligrams Lisinopril and about 20 to 25 milligrams Chlorthalidone in 5 milliliter suspension;
about 35 to 50 milligrams Lisinopril and about 25 to 30 milligrams Chlorthalidone in 5 milliliter suspension;
about 35 to 50 milligrams Lisinopril and about 30 to 40 milligrams Chlorthalidone in 5 milliliter suspension;
about 35 to 50 milligrams Lisinopril and about 40 to 50 milligrams Chlorthalidone in 5 milliliter suspension;
about 35 to 50 milligrams Lisinopril and about 65 to 80 milligrams Chlorthalidone in 5 milliliter suspension;
about 35 to 50 milligrams Lisinopril and about 80 to 100 milligrams Chlorthalidone in 5 milliliter suspension;
about 50 to 65 milligrams Lisinopril and about 10 to 15 milligrams Chlorthalidone in 5 milliliter suspension;
about 50 to 65 milligrams Lisinopril and about 15 to 20 milligrams Chlorthalidone in 5 milliliter suspension;
about 50 to 65 milligrams Lisinopril and about 20 to 25 milligrams Chlorthalidone in 5 milliliter suspension;
about 50 to 65 milligrams Lisinopril and about 25 to 30 milligrams Chlorthalidone in 5 milliliter suspension;
about 50 to 65 milligrams Lisinopril and about 30 to 40 milligrams Chlorthalidone in 5 milliliter suspension;
about 50 to 65 milligrams Lisinopril and about 40 to 50 milligrams Chlorthalidone in 5 milliliter suspension;
about 50 to 65 milligrams Lisinopril and about 50 to 65 milligrams Chlorthalidone in 5 milliliter suspension;
about 50 to 65 milligrams Lisinopril and about 65 to 80 milligrams Chlorthalidone in 5 milliliter suspension; and
about 50 to 65 milligrams Lisinopril and about 80 to 100 milligrams Chlorthalidone in 5 milliliter suspension.

Another liquid dosage form is selected from the group consisting of:

about 2 to 5 milligrams Lisinopril and about 2 to 5 milligrams Chlorthalidone in 5 milliliter suspension;
about 2 to 5 milligrams Lisinopril and about 5 to 10 milligrams Chlorthalidone in 5 milliliter suspension;
about 5 to 10 milligrams Lisinopril and about 10 to 15 milligrams Chlorthalidone in 5 milliliter suspension;
about 10 to 15 milligrams Lisinopril and about 20 to 25 milligrams Chlorthalidone in 5 milliliter suspension;
about 15 to 20 milligrams Lisinopril and about 20 to 25 milligrams Chlorthalidone in 5 milliliter suspension;
about 20 to 25 milligrams Lisinopril and about 25 to 30 milligrams Chlorthalidone in 5 milliliter suspension;
about 25 to 35 milligrams Lisinopril and about 30 to 40 milligrams Chlorthalidone in 5 milliliter suspension;
and about 35 to 50 milligrams Lisinopril and about 40 to 50 milligrams Chlorthalidone in 5 milliliter suspension.

Still another liquid dosage form is selected from the group:

about 5 milligrams Lisinopril and about 6.25 milligrams Chlorthalidone in 5 milliliter suspension;
about 20 milligrams Lisinopril and about 12.5 milligrams Chlorthalidone in 5 milliliter suspension;
about 40 milligrams Lisinopril and about 15 milligrams Chlorthalidone in 5 milliliter suspension;
and about 40 milligrams Lisinopril and about 25 milligrams Chlorthalidone in 5 milliliter suspension.

### Experimental: Liquid Dosage Form

A 5 ml suspension containing 5 mg lisinopril and 6.25 mg chlorthalidone was prepared by the following process:

400 kg sugar was dissolved in 300 liters of purified water and filtered through a 100 mesh filter into a tank. Then a citric acid buffer was prepared using 1 kg citric acid and 4.5 kg sodium citrate in 20 L purified water and added to the tank. 100 kg glycerin and 4 kg sodium carboxy methylcellulose were stirred together and added to the tank. 1.1 kg of lisinopril dihydrate USP was suspended in 20 L purified water and filtered through a 100 mesh filter into the tank. A suspension of 40 L purified water, 10 kg microcrystalline cellulose and 1.25 kg chlorthalidone USP was prepared and filtered through a 40 mesh filter into the tank containing all the other ingredients. 6 kg of strawberry flavor was added although any other suitable flavor could be used. If needed, add purified water to make 1000 L final suspension. The final pH should be between 5 and 6.5; and the final suspension having the appearance of a white colored suspension is ready to be filled into suitable bottles.

A 5 ml suspension containing 20 mg lisinopril and 12.5 chlorthalidone was prepared by the following process:

400 kg sugar was dissolved in 300 liters of purified water and filtered through a 100 mesh filter into a tank. Then a citric acid buffer was prepared using 1 kg citric acid and 4.5 kg sodium citrate in 20 L purified water and added to the tank. 100 kg glycerin and 4 kg sodium carboxy methylcellulose were stirred together and added to the tank. 4.40 kg of lisinopril dihydrate USP was suspended in 20 L purified water and filtered through a 100 mesh filter into the tank. A suspension of 40 L purified water, 10 kg microcrystalline cellulose and 2.50 kg chlorthalidone USP was prepared and filtered through a 40 mesh filter into the tank containing all the other ingredients. 6 kg of strawberry flavor was added although any other suitable flavor could be used. If needed, add purified water to make 1000 L final suspension. The final pH should be between 5 and 6.5 and the final suspension having the appearance of a white colored suspension is ready to be filled into suitable bottles.

A 5 ml suspension containing 40 mg lisinopril and 15 mg chlorthalidone was prepared by the following process:

400 kg sugar was dissolved in 300 liters of purified water and filtered through a 100 mesh filter into a tank. Then a citric acid buffer was prepared using 1 kg citric acid and 4.5 kg sodium citrate in 20 L purified water and added to the tank. 100 kg glycerin and 4 kg sodium carboxy methylcellulose were stirred together and added to the tank. 8.80 kg of lisinopril dihydrate USP was suspended in 20 L purified water and filtered through a 100 mesh filter into the tank. A suspension of 40 L purified water, 10 kg microcrystalline cellulose and 3.00 kg chlorthalidone USP was prepared and filtered through a 40 mesh filter into the tank containing all the other ingredients. 6 kg of strawberry flavor was added although any other suitable flavor could be used. If needed, add purified water to make 1000 L final suspension. The final pH should be between 5 and 6.5 and the final suspension having the appearance of a white colored suspension is ready to be filled into suitable bottles.

A 5 ml suspension containing 40 mg lisinopril and 25 mg chlorthalidone was prepared by the following process:

400 kg sugar was dissolved in 300 liters of purified water and filtered through a 100 mesh filter into a tank. Then a citric acid buffer was prepared using 1 kg citric acid and 4.5 kg sodium citrate in 20 L purified water and added to the tank. 100 kg glycerin and 4 kg sodium carboxy methylcellulose were stirred together and added to the tank. 8.80 kg of lisinopril dihydrate USP was suspended in 20 L purified water and filtered through a 100 mesh filter into the tank. A suspension of 40 L purified water, 10 kg microcrystalline cellulose and 5.00 kg chlorthalidone USP was prepared and filtered through a 40 mesh filter into the tank containing all the other ingredients. 6 kg of strawberry flavor was added although any other suitable flavor could be used. If needed, add purified water to make 1000 L final suspension. The final pH should be between 5 and 6.5 and the final suspension having the appearance of a white colored suspension is ready to be filled into suitable bottles.
In all cases, the oral syrup had the specified concentration of Lisinopril and Chlorthalidone as well as a pharmaceutically acceptable pH range. The suspension was also surprisingly shelf stable as well.

### Experimental: Tablet Dosage Form

Tablets were prepared in two different ways:
Dose Proportional—where the weight proportionality is maintained so that the granules are exactly the same but the compression weights are proportioned in terms of the active concentration required per tablet.
Dose Similar—where the excipients are the same but the concentrations are not in weight proportioned basis but are based on the need of the individual tablet size and weight desired. However, the ratios of excipients remain the same and hence it is dose similar but not necessarily dose proportional.

Dose proportional: 5 mg lisinopril and 6.25 mg chlorthalidone tablets.

0.550 kg of lisinopril dihydrate USP was sifted through a 40 mesh screen, 2.000 kg of D-Mannitol was sifted through a 20 mesh screen and the lisinopril and mannitol were then blended together in a double cone blender. 0.625 kg of chlorthalidone was sifted through a 40 mesh screen and 1.920 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 2.825 kg of sodium starch glycollate was sifted through a 100 mesh screen and then added to the lisinopril-chlorthalidone mixture and the resulting mixture was then blended together. 0.080 kg of magnesium stearate was sifted through an 80 mesh screen and then added to the blended lisinopril-chlorthalidone mixture. After blending, the mixture was compressed in a double rotary compression machine to make 100,000 tablets with an 80 mg tablet weight, each containing 5 mg lisinopril and 6.25 mg chlorthalidone.

Dose proportional: 10 mg lisinopril and 6.25 mg chlorthalidone tablets.

1.100 kg of lisinopril dihydrate USP was sifted through a 40 mesh screen, 4,000 kg of D-Mannitol was sifted through a 20 mesh screen and the lisinopril and mannitol were then blended together in a double cone blender. 0.625 kg of chlorthalidone was sifted through a 40 mesh screen and 3.840 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 5.650 kg of sodium starch glycollate was sifted through a 100 mesh screen and then added to the lisinopril-chlorthalidone mixture and the resulting mixture was then blended together. 0.160 kg of magnesium stearate was sifted through an 80 mesh screen and then added to the blended lisinopril-chlorthalidone mixture. After blending, the mixture was compressed in a double rotary compression machine to make 100,000 tablets with a 153.75 mg tablet weight, each containing 10 mg lisinopril and 6.25 mg chlorthalidone.

Dose proportional: 30 mg lisinopril and 12.5 mg chlorthalidone tablets.

3.300 kg of lisinopril dihydrate USP was sifted through a 40 mesh screen, 12.000 kg of D-Mannitol was sifted through a 20 mesh screen and the lisinopril and mannitol were then blended together in a double cone blender. 1.250 kg of chlorthalidone was sifted through a 40 mesh screen and 11.520 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 16.950 kg of sodium starch glycollate was sifted through a 100 mesh screen and then added to the lisinopril-chlorthalidone mixture and the resulting mixture was then blended together. 0.480 kg of magnesium stearate was sifted through an 80 mesh screen and then added to the blended lisinopril-chlorthalidone mixture. After blending, the mixture was compressed in a double rotary compression machine to make 100,000 tablets with a 455.00 mg tablet weight, each containing 30 mg lisinopril and 12.5 mg chlorthalidone.

Dose proportional: 40 mg lisinopril and 15 mg chlorthalidone tablets.

4.400 kg of lisinopril dihydrate USP was sifted through a 40 mesh screen, 16.000 kg of D-Mannitol was sifted through a 20 mesh screen and the lisinopril and mannitol were then blended together in a double cone blender. 1.500 kg of chlorthalidone was sifted through a 40 mesh screen and 15.360 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 22.600 kg of sodium starch glycollate was sifted through a 100 mesh screen and then added to the lisinopril-chlorthalidone mixture and the resulting mixture was then blended together. 0.640 kg of magnesium stearate was sifted through an 80 mesh screen and then added to the blended lisinopril-chlorthalidone mixture. After blending, the mixture was compressed in a double rotary compression machine to make 100,000 tablets with a 605.00 mg tablet weight, each containing 40 mg lisinopril and 15 mg chlorthalidone.

Dose proportional: 40 mg lisinopril and 25 mg chlorthalidone tablets.

4.400 kg of lisinopril dihydrate USP was sifted through a 40 mesh screen, 16.000 kg of D-Mannitol was sifted through a 20 mesh screen and the lisinopril and mannitol were then blended together in a double cone blender. 2.500 kg of chlorthalidone was sifted through a 40 mesh screen and 15.360 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 22.600 kg of sodium starch glycollate was sifted through a 100 mesh screen and then added to the lisinopril-chlorthalidone mixture and the resulting mixture was then blended together. 0.640 kg of magnesium stearate was sifted through an 80 mesh screen and then added to the blended lisinopril-chlorthalidone mixture. After blending, the mixture was compressed in a double rotary compression machine to make 100,000 tablets with a 615.00 mg tablet weight, each containing 40 mg lisinopril and 25 mg chlorthalidone.

Dose similar: 5 mg lisinopril and 6.25 mg chlorthalidone tablets.

0.550 kg of lisinopril dihydrate USP was sifted through a 40 mesh screen, 2.000 kg of D-Mannitol was sifted through a 20 mesh screen and the lisinopril and mannitol were then blended together in a double cone blender. 0.625 kg of chlorthalidone was sifted through a 40 mesh screen and 1.920 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 2.825 kg of sodium starch glycollate was sifted through a 100 mesh screen and then added to the lisinopril-chlorthalidone mixture and the resulting mixture was then blended together. 0.080 kg of magnesium stearate was sifted through an 80 mesh screen and then added to the blended lisinopril-chlorthalidone mixture. After blending, the mixture was compressed in a double rotary compression machine to make 100,000 tablets with an 80 mg tablet weight, each containing 5 mg lisinopril and 6.25 mg chlorthalidone.

Dose similar: 10 mg lisinopril and 6.25 mg chlorthalidone tablets.

1.100 kg of lisinopril dihydrate USP was sifted through a 40 mesh screen, 4,000 kg of D-Mannitol was sifted through a 20 mesh screen and the lisinopril and mannitol were then blended together in a double cone blender. 0.625 kg of chlorthalidone was sifted through a 40 mesh screen and 4.465 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 5.650 kg of sodium starch glycollate was sifted through a 100 mesh screen and then added to the lisinopril-chlorthalidone mixture and the resulting mixture was then blended together. 0.160 kg of magnesium stearate was sifted through an 80 mesh screen and then added to the blended lisinopril-chlorthalidone mixture. After blending, the mixture was compressed in a double rotary compression machine to make 100,000 tablets with a 160.00 mg tablet weight, each containing 10 mg lisinopril and 6.25 mg chlorthalidone.

Dose similar: 30 mg lisinopril and 12.5 mg chlorthalidone tablets.

3.300 kg of lisinopril dihydrate USP was sifted through a 40 mesh screen, 5.992 kg of D-Mannitol was sifted through a 20 mesh screen and the lisinopril and mannitol were then blended together in a double cone blender. 1.250 kg of chlorthalidone was sifted through a 40 mesh screen and 5.753 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 8.465 kg of sodium starch glycollate was sifted through a 100 mesh screen and then added to the lisinopril-chlorthalidone mixture and the resulting mixture was then blended together. 0.240 kg of magnesium stearate was sifted through an 80 mesh screen and then added to the blended lisinopril-chlorthalidone mixture. After blending, the mixture was compressed in a double rotary compression machine to make 100,000 tablets with a 250.00 mg tablet weight, each containing 30 mg lisinopril and 12.5 mg chlorthalidone.

Dose similar: 40 mg lisinopril and 15 mg chlorthalidone tablets.

4.400 kg of lisinopril dihydrate USP was sifted through a 40 mesh screen, 8.528 kg of D-Mannitol was sifted through a 20 mesh screen and the lisinopril and mannitol were then blended together in a double cone blender. 1.500 kg of chlorthalidone was sifted through a 40 mesh screen and 8.186 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 12.045 kg of sodium starch glycollate was sifted through a 100 mesh screen and then added to the lisinopril-chlorthalidone mixture and the resulting mixture was then blended together. 0.341 kg of magnesium stearate was sifted through an 80 mesh screen and then added to the blended lisinopril-chlorthalidone mixture. After blending, the mixture was compressed in a double rotary compression machine to make 100,000 tablets with a 350.00 mg tablet weight, each containing 40 mg lisinopril and 15 mg chlorthalidone.

Dose similar: 40 mg lisinopril and 25 mg chlorthalidone tablets.

4.400 kg of lisinopril dihydrate USP was sifted through a 40 mesh screen, 8.234 kg of D-Mannitol was sifted through a 20 mesh screen and the lisinopril and mannitol were then blended together in a double cone blender. 2.500 kg of chlorthalidone was sifted through a 40 mesh screen and 7.905 kg of dicalcium phosphate was sifted through a 20 mesh screen. The chlorthalidone and dicalcium phosphate were then blended together in a double cone blender. The lisinopril-mannitol mixture was then added to the chlorthalidone-dicalcium phosphate mixture. 11.631 kg of sodium starch glycollate was sifted through a 100 mesh screen and then added to the lisinopril-chlorthalidone mixture and the resulting mixture was then blended together. 0.330 kg of magnesium stearate was sifted through an 80 mesh screen and then added to the blended lisinopril-chlorthalidone mixture. After blending, the mixture was compressed in a double rotary compression machine to make 100,000 tablets with a 350.00 mg tablet weight, each containing 40 mg lisinopril and 25 mg chlorthalidone.

Both the dose proportional and the dose similar had suitable pharmaceutical properties in terms of disintegration times, tablet hardness and correct proportion of lisinopril to chlorthalidone throughout the tablet run.

We claim:

1. A pharmaceutically acceptable dosage form comprising of a composition of an Angiotensin Converting Enzyme (ACE) inhibitor and Chlorthalidone with suitable pharmaceutically acceptable excipients, with said Angiotensin Converting Enzyme inhibitor being in the range of about 2 to 100 milligrams per dosage form and said Chlorthalidone being in the range of 2 to 100 milligrams per dosage form.

2. The dosage form of claim 1 being selected from the group consisting of tablets, capsules, pills, dissolving films and dragees.

3. The dosage form of claim 1 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 10 to 15 milligrams Chlorthalidone;
about 10 to 15 milligrams Lisinopril and about 15 to 20 milligrams Chlorthalidone;
about 10 to 15 milligrams Lisinopril and about 20 to 25 milligrams Chlorthalidone;
about 15 to 20 milligrams Lisinopril and about 10 to 15 milligrams Chlorthalidone;
about 15 to 20 milligrams Lisinopril and about 15 to 20 milligrams Chlorthalidone;
about 15 to 20 milligrams Lisinopril and about 20 to 25 milligrams Chlorthalidone;
about 20 to 25 milligrams Lisinopril and about 10 to 15 milligrams Chlorthalidone;
about 20 to 25 milligrams Lisinopril and about 15 to 20 milligrams Chlorthalidone;
about 20 to 25 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 10 to 15 milligrams Chlorthalidone;
about 25 to 35 milligrams Lisinopril and about 15 to 20 milligrams Chlorthalidone;
about 25 to 35 milligrams Lisinopril and about 20 to 25 milligrams Chlorthalidone;
about 25 to 35 milligrams Lisinopril and about 25 to 30 milligrams Chlorthalidone;
about 25 to 35 milligrams Lisinopril and about 30 to 40 milligrams Chlorthalidone;
about 35 to 50 milligrams Lisinopril and about 10 to 15 milligrams Chlorthalidone;
about 35 to 50 milligrams Lisinopril and about 15 to 20 milligrams Chlorthalidone;
about 35 to 50 milligrams Lisinopril and about 20 to 25 milligrams Chlorthalidone;
about 35 to 50 milligrams Lisinopril and about 25 to 30 milligrams Chlorthalidone;
about 35 to 50 milligrams Lisinopril and about 30 to 40 milligrams Chlorthalidone;
about 35 to 50 milligrams Lisinopril and about 40 to 50 milligrams Chlorthalidone;
about 35 to 50 milligrams Lisinopril and about 65 to 80 milligrams Chlorthalidone;
about 35 to 50 milligrams Lisinopril and about 80 to 100 milligrams Chlorthalidone;
about 50 to 65 milligrams Lisinopril and about 10 to 15 milligrams Chlorthalidone;
about 50 to 65 milligrams Lisinopril and about 15 to 20 milligrams Chlorthalidone;
about 50 to 65 milligrams Lisinopril and about 20 to 25 milligrams Chlorthalidone;
about 50 to 65 milligrams Lisinopril and about 25 to 30 milligrams Chlorthalidone;
about 50 to 65 milligrams Lisinopril and about 30 to 40 milligrams Chlorthalidone;
about 50 to 65 milligrams Lisinopril and about 40 to 50 milligrams Chlorthalidone;
about 50 to 65 milligrams Lisinopril and about 50 to 65 milligrams Chlorthalidone;
about 50 to 65 milligrams Lisinopril and about 65 to 80 milligrams Chlorthalidone;
and about 50 to 65 milligrams Lisinopril and about 80 to 100 milligrams Chlorthalidone.

4. The dosage form of claim 3 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 20 to 25 milligrams Chlorthalidone;
about 15 to 20 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 30 to 40 milligrams Chlorthalidone;
and about 35 to 50 milligrams Lisinopril and about 40 to 50 milligrams Chlorthalidone.

5. The tablet of claim 2 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 10 to 15 milligrams Chlorthalidone;
about 10 to 15 milligrams Lisinopril and about 15 to 20 milligrams Chlorthalidone;
about 10 to 15 milligrams Lisinopril and about 20 to 25 milligrams Chlorthalidone;
about 15 to 20 milligrams Lisinopril and about 10 to 15 milligrams Chlorthalidone;
about 15 to 20 milligrams Lisinopril and about 15 to 20 milligrams Chlorthalidone;
about 15 to 20 milligrams Lisinopril and about 20 to 25 milligrams Chlorthalidone;
about 20 to 25 milligrams Lisinopril and about 10 to 15 milligrams Chlorthalidone;
about 20 to 25 milligrams Lisinopril and about 15 to 20 milligrams Chlorthalidone;
about 20 to 25 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 10 to 15 milligrams Chlorthalidone;
about 25 to 35 milligrams Lisinopril and about 15 to 20 milligrams Chlorthalidone;
about 25 to 35 milligrams Lisinopril and about 20 to 25 milligrams Chlorthalidone;
about 25 to 35 milligrams Lisinopril and about 25 to 30 milligrams Chlorthalidone;
about 25 to 35 milligrams Lisinopril and about 30 to 40 milligrams Chlorthalidone;
about 35 to 50 milligrams Lisinopril and about 10 to 15 milligrams Chlorthalidone;
about 35 to 50 milligrams Lisinopril and about 15 to 20 milligrams Chlorthalidone;
about 35 to 50 milligrams Lisinopril and about 20 to 25 milligrams Chlorthalidone;
about 35 to 50 milligrams Lisinopril and about 25 to 30 milligrams Chlorthalidone;
about 35 to 50 milligrams Lisinopril and about 30 to 40 milligrams Chlorthalidone;
about 35 to 50 milligrams Lisinopril and about 40 to 50 milligrams Chlorthalidone;
about 35 to 50 milligrams Lisinopril and about 65 to 80 milligrams Chlorthalidone;
about 35 to 50 milligrams Lisinopril and about 80 to 100 milligrams Chlorthalidone;
about 50 to 65 milligrams Lisinopril and about 10 to 15 milligrams Chlorthalidone;
about 50 to 65 milligrams Lisinopril and about 15 to 20 milligrams Chlorthalidone;
about 50 to 65 milligrams Lisinopril and about 20 to 25 milligrams Chlorthalidone;
about 50 to 65 milligrams Lisinopril and about 25 to 30 milligrams Chlorthalidone;
about 50 to 65 milligrams Lisinopril and about 30 to 40 milligrams Chlorthalidone;
about 50 to 65 milligrams Lisinopril and about 40 to 50 milligrams Chlorthalidone;
about 50 to 65 milligrams Lisinopril and about 50 to 65 milligrams Chlorthalidone.
about 50 to 65 milligrams Lisinopril and about 65 to 80 milligrams Chlorthalidone;
and about 50 to 65 milligrams Lisinopril and about 80 to 100 milligrams Chlorthalidone;

6. The tablet of claim 5 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 20 to 25 milligrams Chlorthalidone;
about 15 to 20 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 30 to 40 milligrams Chlorthalidone;
and about 35 to 50 milligrams Lisinopril and about 40 to 50 milligrams Chlorthalidone.

7. The capsule of claim 2 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 10 to 15 milligrams Chlorthalidone;
about 10 to 15 milligrams Lisinopril and about 15 to 20 milligrams Chlorthalidone;
about 10 to 15 milligrams Lisinopril and about 20 to 25 milligrams Chlorthalidone;
about 15 to 20 milligrams Lisinopril and about 10 to 15 milligrams Chlorthalidone;
about 15 to 20 milligrams Lisinopril and about 15 to 20 milligrams Chlorthalidone;
about 15 to 20 milligrams Lisinopril and about 20 to 25 milligrams Chlorthalidone;
about 20 to 25 milligrams Lisinopril and about 10 to 15 milligrams Chlorthalidone;
about 20 to 25 milligrams Lisinopril and about 15 to 20 milligrams Chlorthalidone;
about 20 to 25 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 10 to 15 milligrams Chlorthalidone;
about 25 to 35 milligrams Lisinopril and about 15 to 20 milligrams Chlorthalidone;
about 25 to 35 milligrams Lisinopril and about 20 to 25 milligrams Chlorthalidone;
about 25 to 35 milligrams Lisinopril and about 25 to 30 milligrams Chlorthalidone;
about 25 to 35 milligrams Lisinopril and about 30 to 40 milligrams Chlorthalidone;
about 35 to 50 milligrams Lisinopril and about 10 to 15 milligrams Chlorthalidone;
about 35 to 50 milligrams Lisinopril and about 15 to 20 milligrams Chlorthalidone;
about 35 to 50 milligrams Lisinopril and about 20 to 25 milligrams Chlorthalidone;
about 35 to 50 milligrams Lisinopril and about 25 to 30 milligrams Chlorthalidone;
about 35 to 50 milligrams Lisinopril and about 30 to 40 milligrams Chlorthalidone;
about 35 to 50 milligrams Lisinopril and about 40 to 50 milligrams Chlorthalidone;
about 35 to 50 milligrams Lisinopril and about 65 to 80 milligrams Chlorthalidone;
about 35 to 50 milligrams Lisinopril and about 80 to 100 milligrams Chlorthalidone;
about 50 to 65 milligrams Lisinopril and about 10 to 15 milligrams Chlorthalidone;
about 50 to 65 milligrams Lisinopril and about 15 to 20 milligrams Chlorthalidone;
about 50 to 65 milligrams Lisinopril and about 20 to 25 milligrams Chlorthalidone;
about 50 to 65 milligrams Lisinopril and about 25 to 30 milligrams Chlorthalidone;
about 50 to 65 milligrams Lisinopril and about 30 to 40 milligrams Chlorthalidone;
about 50 to 65 milligrams Lisinopril and about 40 to 50 milligrams Chlorthalidone;
about 50 to 65 milligrams Lisinopril and about 50 to 65 milligrams Chlorthalidone.
about 50 to 65 milligrams Lisinopril and about 65 to 80 milligrams Chlorthalidone;
and about 50 to 65 milligrams Lisinopril and about 80 to 100 milligrams Chlorthalidone;

8. The capsule of claim 7 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 20 to 25 milligrams Chlorthalidone;
about 15 to 20 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 30 to 40 milligrams Chlorthalidone;
and about 35 to 50 milligrams Lisinopril and about 40 to 50 milligrams Chlorthalidone.

9. The dosage form of claim 4 wherein the compositions selected from the group consisting of:
about 2.5 milligrams Lisinopril and 6.25 milligrams Chlorthalidone;
about 5 milligrams Lisinopril and about 12.5 milligrams Chlorthalidone;
about 10 milligrams Lisinopril and about 25 milligrams Chlorthalidone;
about 20 milligrams Lisinopril and about 50 milligrams Chlorthalidone;
and about 40 milligrams Lisinopril and about 100 milligrams Chlorthalidone.

10. The tablet of claim 6 wherein the composition is selected from the group consisting of:
about 2.5 milligrams Lisinopril and 6.25 milligrams Chlorthalidone;
about 5 milligrams Lisinopril and about 12.5 milligrams Chlorthalidone;
about 10 milligrams Lisinopril and about 25 milligrams Chlorthalidone;
about 20 milligrams Lisinopril and about 50 milligrams Chlorthalidone;
and about 40 milligrams Lisinopril and about 100 milligrams Chlorthalidone.

11. The capsule of claim 8 wherein the composition is selected from the group consisting of:
about 2.5 milligrams Lisinopril and 6.25 milligrams Chlorthalidone;
about 5 milligrams Lisinopril and about 12.5 milligrams Chlorthalidone;
about 10 milligrams Lisinopril and about 25 milligrams Chlorthalidone;
about 20 milligrams Lisinopril and about 50 milligrams Chlorthalidone;
and about 40 milligrams Lisinopril and about 100 milligrams Chlorthalidone.

12. A method of treating hypertension comprising giving a patent in need thereof a pharmaceutically acceptable dosage form comprising a composition of Angiotensin Converting Enzyme (ACE) inhibitor and Chlorthalidone with suitable pharmaceutically acceptable excipients, with said Angiotensin Converting Enzyme inhibitor being in the range of about 2 to 100 milligrams per dosage form and said Chlorthalidone being in the range of 2 to 100 milligrams per dosage form.

13. The method of claim 12 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 10 to 15 milligrams Chlorthalidone;
about 10 to 15 milligrams Lisinopril and about 15 to 20 milligrams Chlorthalidone;
about 10 to 15 milligrams Lisinopril and about 20 to 25 milligrams Chlorthalidone;
about 15 to 20 milligrams Lisinopril and about 10 to 15 milligrams Chlorthalidone;
about 15 to 20 milligrams Lisinopril and about 15 to 20 milligrams Chlorthalidone;
about 15 to 20 milligrams Lisinopril and about 20 to 25 milligrams Chlorthalidone;
about 20 to 25 milligrams Lisinopril and about 10 to 15 milligrams Chlorthalidone;
about 20 to 25 milligrams Lisinopril and about 15 to 20 milligrams Chlorthalidone;
about 20 to 25 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 10 to 15 milligrams Chlorthalidone;
about 25 to 35 milligrams Lisinopril and about 15 to 20 milligrams Chlorthalidone;
about 25 to 35 milligrams Lisinopril and about 20 to 25 milligrams Chlorthalidone;
about 25 to 35 milligrams Lisinopril and about 25 to 30 milligrams Chlorthalidone;
about 25 to 35 milligrams Lisinopril and about 30 to 40 milligrams Chlorthalidone;
about 35 to 50 milligrams Lisinopril and about 10 to 15 milligrams Chlorthalidone;
about 35 to 50 milligrams Lisinopril and about 15 to 20 milligrams Chlorthalidone;
about 35 to 50 milligrams Lisinopril and about 20 to 25 milligrams Chlorthalidone;
about 35 to 50 milligrams Lisinopril and about 25 to 30 milligrams Chlorthalidone;
about 35 to 50 milligrams Lisinopril and about 30 to 40 milligrams Chlorthalidone;
about 35 to 50 milligrams Lisinopril and about 40 to 50 milligrams Chlorthalidone;
about 35 to 50 milligrams Lisinopril and about 65 to 80 milligrams Chlorthalidone;
about 35 to 50 milligrams Lisinopril and about 80 to 100 milligrams Chlorthalidone;
about 50 to 65 milligrams Lisinopril and about 10 to 15 milligrams Chlorthalidone;
about 50 to 65 milligrams Lisinopril and about 15 to 20 milligrams Chlorthalidone;
about 50 to 65 milligrams Lisinopril and about 20 to 25 milligrams Chlorthalidone;
about 50 to 65 milligrams Lisinopril and about 25 to 30 milligrams Chlorthalidone;
about 50 to 65 milligrams Lisinopril and about 30 to 40 milligrams Chlorthalidone;
about 50 to 65 milligrams Lisinopril and about 40 to 50 milligrams Chlorthalidone;
about 50 to 65 milligrams Lisinopril and about 50 to 65 milligrams Chlorthalidone.
about 50 to 65 milligrams Lisinopril and about 65 to 80 milligrams Chlorthalidone;
and about 50 to 65 milligrams Lisinopril and about 80 to 100 milligrams Chlorthalidone.

14. The method of claim 13 wherein the composition is selected from the group consisting of:
about 2 to 5 milligrams Lisinopril and about 2 to 5 milligrams Chlorthalidone;
about 2 to 5 milligrams Lisinopril and about 5 to 10 milligrams Chlorthalidone;
about 5 to 10 milligrams Lisinopril and about 10 to 15 milligrams Chlorthalidone;
about 10 to 15 milligrams Lisinopril and about 20 to 25 milligrams Chlorthalidone;
about 15 to 20 milligrams Lisinopril and about 20 to 25 milligrams Chlorthalidone;
about 20 to 25 milligrams Lisinopril and about 25 to 30 milligrams Chlorthalidone;
about 25 to 35 milligrams Lisinopril and about 30 to 40 milligrams Chlorthalidone;
and about 35 to 50 milligrams Lisinopril and about 40 to 50 milligrams Chlorthalidone.

15. The method of claim 14 wherein the composition is selected from the group consisting of
about 2.5 milligrams Lisinopril and 6.25 milligrams Chlorthalidone;
about 5 milligrams Lisinopril and about 12.5 milligrams Chlorthalidone;
about 10 milligrams Lisinopril and about 25 milligrams Chlorthalidone;
about 20 milligrams Lisinopril and about 50 milligrams Chlorthalidone;
and about 40 milligrams Lisinopril and about 100 milligrams Chlorthalidone.

16. The method of claim 12 wherein the dosage form of claim 12 is being selected from the group consisting of tablets, capsules, pills and dragees.

17. An oral dosage form of a mixture of Angiotensin Converting Enzyme (ACE) inhibitor and Chlorthalidone comprising, on a weight to weight basis, about 1 to 2 parts Lisinopril to every 1 to 4 parts Chlorthalidone in sufficient, pharmaceutically acceptable syrup to provide a pharmaceutically acceptable dose of the Angiotensin Converting Enzyme (ACE) inhibitor-Chlorthalidone combination.

18. The dosage form of claim 17 wherein the dosage form is a stable liquid suspension having a ratio of Lisinopril to Chlorthalidone selected from the group consisting of
about 2 to 5 milligrams Lisinopril and about 2 to 5 milligrams Chlorthalidone in 5 milliliter suspension;
about 2 to 5 milligrams Lisinopril and about 5 to 10 milligrams Chlorthalidone in 5 milliliter suspension;
about 5 to 10 milligrams Lisinopril and about 5 to 10 milligrams Chlorthalidone in 5 milliliter suspension;
about 5 to 10 milligrams Lisinopril and about 10 to 15 milligrams Chlorthalidone in 5 milliliters suspension
about 10 to 15 milligrams Lisinopril and about 10 to 15 milligrams Chlorthalidone in 5 milliliter suspension;
about 10 to 15 milligrams Lisinopril and about 15 to 20 milligrams Chlorthalidone in 5 milliliter suspension;
about 10 to 15 milligrams Lisinopril and about 20 to 25 milligrams Chlorthalidone in 5 milliliter suspension;
about 15 to 20 milligrams Lisinopril and about 10 to 15 milligrams Chlorthalidone in 5 milliliter suspension;
about 15 to 20 milligrams Lisinopril and about 15 to 20 milligrams Chlorthalidone in 5 milliliter suspension;
about 15 to 20 milligrams Lisinopril and about 20 to 25 milligrams Chlorthalidone in 5 milliliter suspension;
about 20 to 25 milligrams Lisinopril and about 10 to 15 milligrams Chlorthalidone in 5 milliliter suspension;
about 20 to 25 milligrams Lisinopril and about 15 to 20 milligrams Chlorthalidone in 5 milliliter suspension;
about 20 to 25 milligrams Lisinopril and about 20 to 25 milligrams Chlorthalidone in 5 milliliter suspension;
about 20 to 25 milligrams Lisinopril and about 25 to 30 milligrams Chlorthalidone in 5 milliliter suspension;
about 25 to 35 milligrams Lisinopril and about 10 to 15 milligrams Chlorthalidone in 5 milliliter suspension;
about 25 to 35 milligrams Lisinopril and about 15 to 20 milligrams Chlorthalidone in 5 milliliter suspension;
about 25 to 35 milligrams Lisinopril and about 20 to 25 milligrams Chlorthalidone in 5 milliliter suspension;
about 25 to 35 milligrams Lisinopril and about 25 to 30 milligrams Chlorthalidone in 5 milliliter suspension;
about 25 to 35 milligrams Lisinopril and about 30 to 40 milligrams Chlorthalidone in 5 milliliter suspension;
about 35 to 50 milligrams Lisinopril and about 10 to 15 milligrams Chlorthalidone in 5 milliliter suspension;
about 35 to 50 milligrams Lisinopril and about 15 to 20 milligrams Chlorthalidone in 5 milliliter suspension;
about 35 to 50 milligrams Lisinopril and about 20 to 25 milligrams Chlorthalidone in 5 milliliter suspension;
about 35 to 50 milligrams Lisinopril and about 25 to 30 milligrams Chlorthalidone in 5 milliliter suspension;
about 35 to 50 milligrams Lisinopril and about 30 to 40 milligrams Chlorthalidone in 5 milliliter suspension;
about 35 to 50 milligrams Lisinopril and about 40 to 50 milligrams Chlorthalidone in 5 milliliter suspension;
about 35 to 50 milligrams Lisinopril and about 65 to 80 milligrams Chlorthalidone in 5 milliliter suspension;
about 35 to 50 milligrams Lisinopril and about 80 to 100 milligrams Chlorthalidone in 5 milliliter suspension;
about 50 to 65 milligrams Lisinopril and about 10 to 15 milligrams Chlorthalidone in 5 milliliter suspension;
about 50 to 65 milligrams Lisinopril and about 15 to 20 milligrams Chlorthalidone in 5 milliliter suspension;
about 50 to 65 milligrams Lisinopril and about 20 to 25 milligrams Chlorthalidone in 5 milliliter suspension;
about 50 to 65 milligrams Lisinopril and about 25 to 30 milligrams Chlorthalidone in 5 milliliter suspension;
about 50 to 65 milligrams Lisinopril and about 30 to 40 milligrams Chlorthalidone in 5 milliliter suspension;
about 50 to 65 milligrams Lisinopril and about 40 to 50 milligrams Chlorthalidone in 5 milliliter suspension;
about 50 to 65 milligrams Lisinopril and about 50 to 65 milligrams Chlorthalidone in 5 milliliter suspension.
about 50 to 65 milligrams Lisinopril and about 65 to 80 milligrams Chlorthalidone in 5 milliliter suspension; and
about 50 to 65 milligrams Lisinopril and about 80 to 100 milligrams Chlorthalidone in 5 milliliter suspension.

19. The dosage form of claim 18 wherein the composition is selected from the group consisting of:
about 2 to 5 milligrams Lisinopril and about 2 to 5 milligrams Chlorthalidone in 5 milliliter suspension;
about 2 to 5 milligrams Lisinopril and about 5 to 10 milligrams Chlorthalidone in 5 milliliter suspension;
about 5 to 10 milligrams Lisinopril and about 10 to 15 milligrams Chlorthalidone in 5 milliliter suspension;
about 10 to 15 milligrams Lisinopril and about 20 to 25 milligrams Chlorthalidone in 5 milliliter suspension;
about 15 to 20 milligrams Lisinopril and about 20 to 25 milligrams Chlorthalidone in 5 milliliter suspension;
about 20 to 25 milligrams Lisinopril and about 25 to 30 milligrams Chlorthalidone in 5 milliliter suspension;
about 25 to 35 milligrams Lisinopril and about 30 to 40 milligrams Chlorthalidone in 5 milliliter suspension;
and about 35 to 50 milligrams Lisinopril and about 40 to 50 milligrams Chlorthalidone in 5 milliliter suspension.

20. The dosage form of claim 19 wherein composition is selected from the group consisting of
about 5 milligrams Lisinopril and about 6.25 milligrams Chlorthalidone in 5 milliliter suspension;
about 20 milligrams Lisinopril and about 12.5 milligrams Chlorthalidone in 5 milliliter suspension;
about 40 milligrams Lisinopril and about 15 milligrams Chlorthalidone in 5 milliliter suspension;
and about 40 milligrams Lisinopril and about 25 milligrams Chlorthalidone in 5 milliliter suspension.
